(19) United States
(12) Patent Application Publication
(10) Pub. No.: US 2024/0207523 A1
(43) Pub. Date: Jun. 27, 2024

(54) Housing Components for an Injection Device

(71) Applicant: Sanofi, Paris (FR)

(72) Inventors: Beate Franke, Frankfurt am Main (DE); Christian Frescha, Rüsselsheim (DE); Michael Helmer, Frankfurt am Main (DE); Stephan Muecke, Rüsselsheim (DE); Peter Nober, Rüsselsheim (DE); Matthias Rau, Rüsselsheim (DE); Michael Schabbach, Frankfurt am Main (DE); Martin Vitt, Frankfurt am Main (DE)

(21) Appl. No.: 18/288,449

(22) PCT Filed: May 2, 2022
(86) PCT No.: PCT/EP2022/061646

§ 371 Date: Oct. 26, 2023

(30) Foreign Application Priority Data
May 3, 2021 (EP) 21315076.6

(51) Int. Cl. A61M 5/24; A61M 5/31; A61M 5/315

(52) U.S. Cl. CPC A61M 5/2459; A61M 5/31593; A61M 2005/2462; A61M 2005/3126; A61M 2205/6036; A61M 2205/6045

(57) ABSTRACT

A drug delivery housing device includes a first housing component comprising a first connecting end, and a second housing component comprising a second connecting end. An insert is provided on the first or second connecting end, and a receptacle is provided on the other one of the first and second connecting ends. The insert is insertable into the receptacle for mutually fastening the first and second housing components. A threaded connection mutually connects the first and second connecting ends and includes a helical thread on the insert and a helical counter thread complementary shaped to the helical thread and provided in the receptacle. A rotation lock is configured to impede screwing and/or unscrewing of the first and second housing components. The rotation lock includes a locking structure on the first housing component and a counter locking structure complementary shaped to the locking structure and provided on the second housing component.

### CROSS REFERENCE TO RELATED APPLICATIONS

The present application is the national stage entry of International Patent Application No. PCT/EP2022/061646, filed on May 2, 2022, and claims priority to Application No. EP 21315076.6, filed on May 3, 2021, the disclosures of which are incorporated herein by reference.

### TECHNICAL FIELD

The present disclosure relates to the field of drug delivery devices and systems, particularly to injection devices for injecting a liquid medicament. More particularly, the present disclosure is generally directed to drug delivery devices and systems comprising a multi-component housing, wherein one housing component is configured to accommodate a medicament container, such as a cartridge and wherein another housing component is configured to accommodate a drive mechanism to operably engage with the medicament container for expelling or withdrawing a dose of the medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump. With respect to reusable injection or delivery devices, a patient may have to load or to replace a cartridge. Reusable injection devices typically comprise a multi-component housing. For instance, the housing may comprise a proximal housing component, such as a body and a distal housing component, such as a cartridge holder detachably connectable to the body. Once a medicament provided in a medicament container, such as a cartridge, is empty, the cartridge holder may be disconnected from the body of the injection device and the empty cartridge may be removed and replaced with a new cartridge.

Another concern may arise from cartridges being manufactured in essentially standard sizes and manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but fitting a single drug delivery device. As just one example, a first cartridge containing a first medicament from a first supplier may fit a drug delivery device provided by a second supplier. As such, a user might be able to load an incorrect medicament into a drug delivery device and, then, dispense said medicament (such as a rapid or basal type of insulin) without being aware that the medical delivery device was perhaps neither designed nor intended to be used with such a cartridge.

As such, there is a growing desire from users, health care providers, caregivers, regulatory entities, and medical device suppliers to reduce the potential risk of a user loading an incorrect drug type into a drug delivery device. It is also desirable to reduce the risk of dispensing an incorrect medicament (or the wrong concentration of the medicament) from such a drug delivery device.

There is, therefore, a general need to physically dedicate or mechanically code a cartridge and/or cartridge holder to its drug type and design an injection device that only accepts or works with the dedication or coded features provided on or with the cartridge and/or cartridge holder so as to prevent unwanted cartridge cross use. Similarly, there is also a general need for a dedicated cartridge that allows the medical delivery device to be used with only an authorized cartridge containing a specific medicament while also preventing undesired cartridge cross use.

With drug delivery devices comprising a multi-component housing, e.g. having a first and a second housing component, it is also desirable to provide a failure safe and well-defined mechanical connection between the housing components, which may be detachably or non-detachably connectable. Here, the present disclosure aims to provide an improvement for connecting and disconnecting first and second housing components of a drug delivery device.

### SUMMARY

In one aspect the disclosure relates to a housing of a drug delivery device, in particular to a housing of an injection device, such as a handheld injection pen. The housing comprises a first housing component configured to accommodate a cartridge filled with a medicament. The first housing component comprises a first connecting end. The housing further comprises a second housing component. The second housing component is configured to accommodate a drive mechanism of the drug delivery device. Typically, the drive mechanism comprises a piston rod extending in longitudinal direction and configured to operably engage with a piston or bung of the cartridge for expelling a dose of the medicament from the cartridge.

The second housing component comprises a second connecting end. Typically, the first connecting end is connectable to the second connecting end to form or to constitute the housing of the drug delivery device. With some examples the first housing component is an elongated or tubular shaped housing component comprising the first connecting end at a longitudinal proximal end. The second housing component may be also of tubular or elongated shape. The second connecting end may be located at a distal longitudinal end of the second housing component.

There is further provided an insert on one of the first connecting end and the second connecting end. The insert is typically integrally formed with the respective first or second housing component. There is further provided a receptacle on the other one of the first connecting end and the second connecting end. The insert is insertable into the receptacle along the longitudinal direction for mutually fastening the first housing component and the second housing component and/or for forming or establishing the housing of the drug delivery device. Typically, the receptacle is provided at one of the first and second connecting ends and forms a respective connecting end. The insert is provided on the other one of the first and second connecting ends and forms a respective connecting end.

The receptacle comprises an inner cross-section sized and shaped to receive the insert therein. Typically, an inside diameter or inside cross-section of the receptacle closely matches an outside diameter or outer cross-section of the insert.

The housing may further comprise a fastening element provided on the insert and a counter fastening element complementary shaped to the fastening element and provided in the receptacle. Typically, and when reaching a final assembly configuration, the fastening element engages the counter fastening element thereby fastening and fixing the first housing component to the second housing component; and vice versa.

The housing further comprises a threaded connection to mutually connect and/or to mutually fix the first connecting end and the second connecting end. The threaded connection comprises a helical thread provided on the insert. The threaded connection further comprises a helical counter thread complementary shaped to the helical thread and provided in the receptacle. The helical thread is typically provided on an outside surface of the insert. The helical counter thread is typically provided on an inside surface of a sidewall of the receptacle. Typically, one of the helical thread and the helical counter thread comprises a radially protruding rib and the other one of the helical thread and the helical counter thread comprises a radially recessed groove complementary shaped to the radially protruding rib.

With some examples, at least one of the helical thread and the helical counter thread comprises an angular width of at least 360°. Hence, the threaded connection comprises at least one or more revolutions,

The fastening element of the insert may be provided by the helical thread and the counter fastening element of the receptacle may be provided by the helical counter thread.

The housing further comprises a rotation lock configured or operable to impede screwing and/or unscrewing of the first housing component and the second housing component when in a final assembly configuration. The rotation lock comprises a locking structure on the first housing component and a counter locking structure complementary shaped to the locking structure and provided on the second housing component.

The rotation lock may be configured to impede or to prevent a further screwing down of the first housing component and the second housing component when or after reaching a final assembly configuration. The rotation lock may be also configured to impede unscrewing of the first housing component and the second housing component when reaching or upon reaching the final assembly configuration. In the final assembly configuration, the first housing component is fixed to the second housing component; and vice versa. In the final assembly configuration, the insert is received and accommodated in the receptacle. Typically, the insert is entirely received inside the receptacle.

During or for mutual assembly of the first and second housing components the insert of one of the first and second housing components is longitudinally inserted into the receptacle of the other one of the first and second housing components, e.g. by way of a longitudinally sliding motion. During or after inserting of the insert into the receptacle the helical thread provided on the insert engages the helical counter thread. Thereafter, the mutual assembly or fastening of the first and second housing component is governed by a helical screw motion of the first housing component relative to the second housing component. The screw type fastening is defined by the geometry, shape and by the pitch of the thread and the complementary-shaped counter thread.

The final assembly configuration is finally reached by screwing down the first and the second housing components until the rotation lock is activated. Activation of the rotation lock includes a mechanical engagement of the locking structure of the first housing component with the complementary shaped counter locking structure of the second housing component.

When mutually engaged the locking structure and the counter locking structure of the rotation lock are configured and shaped to transfer a torque from the first housing component to the second housing component; and vice versa. Hence, when the locking structure engages the counter locking structure a rotation of the first housing component relative to the second housing component is effectively blocked and/or impeded.

The rotation lock is particularly configured to prevent a further screwing down of the first housing component relative to the second housing component when or after a final assembly configuration has been reached. In this way, over tightening of the threaded connection between the first and second housing component can be effectively prevented. This has the benefit to prevent an unintended damage of the first and/or second housing component upon establishing of the screw type interconnection between the first and second housing components.

Additionally or alternatively, the rotation lock may also prevent or impede unscrewing of the first housing component and the second housing component once the housing components are in the final assembly configuration. In this way an unintended unscrewing of first and second housing components can be effectively prevented.

The locking structure of the first housing component and the counter locking structure of the second housing component may audibly and/or haptically engage. In this way an audible and/or haptic feedback is generated thus indicating to a user, that a well-defined final assembly configuration has been reached. By way of a palpable or audible engagement of the locking structure and the counter locking structure the user is prompted to stop screwing down the first and second housing components for connecting and/or fixing the same relative to each other. The palpable or audible engagement therefore provides a well-defined feedback to the user that the final assembly configuration has been reached and that a further screwing down of the first and the second housing component is not required any longer.

According to a further example the locking structure and the counter locking structure of the rotation lock are mutually engageable through a twisting helical motion of the first housing component relative to the second housing component upon or during reaching the final assembly configuration of the first and second housing components. The locking structure is particularly shaped and configured to allow and to support an engagement with the counter locking structure through a twisting helical motion. Typically, the twisting helical motion is defined by the threaded connection of the first connecting end and the second connecting end of the first and second housing components, respectively. In this way, the mutual engagement of the locking structure with the counter locking structure takes place or is established at the very end of the screw type fastening of the first connecting end and the second connecting end, which is defined by the helical thread of the insert and the complementary or correspondingly shaped helical counter thread of the receptacle.

The mutual engaging of the locking structure with the counter locking structure is simply obtained by a mutual helical guiding of the insert and the receptacle as defined by the helical thread and the complementary shaped helical counter thread. The ability of the locking structure to engage with the counter locking structure through a twisting or rotating helical motion is therefore beneficial to combine the rotation lock with the threaded connection of the housing.

According to a further example the locking structure provided on the first housing component and the counter locking structure provided on the second housing component may be configured to mechanically engage by way of a snap fit engagement. In other words, the mutual engagement of the locking structure and the counter locking structure may include or may provide a snap fit engagement.

During or for a mutual engagement of the first and second housing components at least one of the locking structure and the counter locking structure may be subject to an elastic deformation. When reaching the final assembly configuration an elastically deformed portion of the locking structure and/or the counter locking structure may be allowed to relax into an initial state, such that the locking structure and the counter locking structure establish or contribute to a form fit engagement between the first housing component and the second housing component. The form fit established or provided by the locking structure engaged with the counter locking structure may be configured and hence operable to transfer a torque from the first housing component to the second housing component; and vice versa. Thus, the mutual engagement between the locking structure and the counter locking structure may be of a torque transmitting type.

According to a further example the locking structure comprises a toothed profile complementary shaped to a counter toothed profile of the counter locking structure. One of the toothed profile and the counter toothed profile comprises at least one locking tooth extending in longitudinal direction. The other one of the toothed profile and the counter toothed profile comprises at least one locking recess complementary shaped to the locking tooth. The longitudinally or axially extending locking tooth and the complementary shaped longitudinally or axially extending locking recess provide a torque transmitting engagement between the toothed profile and the counter toothed profile and hence a torque transmitting engagement of the locking structure and the counter locking structure, at least when the first and the second housing components have reached the final assembly configuration.

Typically, the longitudinal extent as well as the size and shape of the at least one locking tooth matches the respective longitudinal or axial extent, size and the shape or profile of the complementary shaped locking recess. When reaching the final assembly configuration, the entirety of the locking tooth is typically received in the locking recess. In this way a flank of the locking tooth may engage or abut with a complementary-shaped flank of the locking recess, thereby providing a torque transmitting mechanical engagement between the locking structure and the counter locking structure.

According to a further example the at least one locking tooth comprises an asymmetric profile with regard to a circumferential direction of the first housing component or second housing component. The asymmetric profile in circumferential or tangential direction is configured and/or shaped to implement different forces or torques for establishing and releasing a mutual engagement between the locking structure and the counter locking structure for connecting and/or disconnecting the first and second housing components.

The asymmetric profile of the at least one locking tooth may require and define an interlock force that must be overcome during mutual assembly of the first and second housing components, in particular during and for reaching the final assembly configuration. The asymmetric profile of the at least one locking tooth may further define a release force that must be overcome or overruled to abrogate or release the final assembly configuration, e.g. for disconnecting the first and the second housing components from each other. In other words, the asymmetric profile of the at least one locking tooth defines an interlock force that must be provided by a user in order to engage the locking structure and the counter locking structure to reach the final assembly configuration. The at least one locking tooth further defines a release force required for disengaging the locking structure from the counter locking structure when the first and second housing components are in the final assembly configuration, hence when the locking structure and the counter locking structure are mutually engaged.

With some typical examples the at least one locking tooth and its asymmetric profile is designed and configured such that the interlock force is smaller than the release force. Insofar, a force or torque required for establishing a mutual engagement between the locking structure and the counter locking structure is less than a force or torque required for disconnecting or disengaging the locking structure and the counter locking structure. In this way, the asymmetric structure of the rotation lock helps to prevent an unintended disconnection of the first and the second housing components when in the final assembly configuration.

With some other examples the asymmetric profile may provide a release force that is smaller than an interlock force.

According to a further example the at least one locking recess comprises an asymmetric profile with regard to a circumferential direction of the first housing component or second housing component. Typically, the asymmetric profile of the at least one locking recess is complementary shaped to the asymmetric profile of the at least one locking tooth. In this way both side edges or flanks of the locking tooth may engage with complementary or correspondingly shaped side edges or flanks of the locking recess.

According to a further example the at least one locking tooth comprises a first tooth flank facing in a first circumferential direction. The at least one locking tooth further comprises a second tooth flank facing in a second circumferential direction opposite the first circumferential direction. According to a further example the first tooth flank distinguishes from the second tooth flank by at least one of a circumferential length and a flank angle. With some examples the first tooth flank distinguishes from the second tooth flank by both, the flank angle and by the circumferential length. With other examples it is only the flank angle or the circumferential length of the first tooth flank that distinguishes from the respective flank angle or circumferential length of the second tooth flank.

The first tooth flank and the second tooth flank distinguish from each other when the at least one locking tooth comprises an asymmetric profile with regard to a circumferential direction. When the first tooth flank and the second tooth flank distinguish from each other it is particularly the size of the flank angle of the first tooth flank that distinguishes from the size of the flank angle of the second tooth flank. In this way the respective flanks do not only distinguish by their direction with regard to the longitudinal axis or the circumferential direction but also by the size of the angle. In other words, the steepness of the first tooth flank distinguishes from the steepness of the second tooth flank.

By way of differently sized flank angles and/or by way of different circumferential extents of first and second tooth flanks a well-defined asymmetric profile of the at least one locking tooth can be provided in order to implement differently sized interlock and release forces for establishing and for releasing the final assembly configuration of the threaded connection of the first and second housing components.

According to a further example the circumferential length of the first tooth flank is larger than the circumferential length of the second tooth flank. Moreover, the flank angle of the first tooth flank is smaller than the flank angle of the second tooth flank. With some examples a bottom portion of the first tooth flank and a bottom portion of the second tooth flank are located at the same longitudinal position on the first or second housing component. With other examples a bottom section of the first tooth flank and a bottom section of the second tooth flank are located at different longitudinal position. They may be longitudinally or axially staggered.

According to a further example the at least one locking recess comprises a first flank facing in a first circumferential direction and a second flank facing in a second circumferential direction opposite the first circumferential direction. With some examples the first flank distinguishes from the second flank by at least one of a circumferential length and a flank angle. The first flank may distinguish from the second flank by both, its circumferential length and its flank angle. Hence, the circumferential length and the flank angle of the first flank may distinguish from the circumferential length and the flank angle of the second flank.

Typically, the at least one locking recess comprises the same asymmetric profile than the at least one locking tooth. The at least one locking recess is complementary shaped to the at least one locking tooth.

According to a further example the circumferential length of the first flank is larger than the circumferential length of the second flank. Moreover, the flank angle of the first flank is smaller than the flank angle of the second flank. Accordingly, and since the at least one locking recess is complementary or correspondingly shaped to the at least one locking tooth all features and benefits as described above with regards to the locking tooth equally apply to the complementary shaped locking recess.

According to a further example the circumferential length of the first tooth flank is substantially equal to the circumferential length of the first flank of the at least one locking recess. The same may apply to the flank angle. Hence, the flank angle of the first tooth flank may be substantially identical or equivalent to the flank angle of the first flank of the at least one locking recess. The same may also apply to the second flank with regard to the circumferential extent as well as with regard to the flank angle. In particular, the circumferential extent of the second flank of the at least one locking tooth may be identical or equivalent to the circumferential extent of the second flank of the at least one locking recess. Also, the flank angle of the second flank of the at least one locking tooth may be substantially equal to or may match the flank angle of the second flank of the at least one locking recess.

In this way and when reaching a final assembly configuration, the first flank of the at least one locking tooth may be aligned and/or in abutment with the first flank of the complementary shaped locking recess. The same may apply to the second flank. When in the final assembly configuration, the second flank of the at least one locking tooth may engage or may align the second flank of the complementary shaped locking recess.

According to another example the flank angle of the first tooth flank and/or the flank angle of the first flank is smaller than or equal the thread pitch of the helical thread or helical counter thread of the threaded connection. In this way a rather smooth and easy sliding engagement of the toothed profile and the counter toothed profile for screwing down the threaded connection and/or for establishing the threaded connection can be implemented and provided. Hence, a rather low interlock force or interlocked pork for establishing the final assembly configuration of the first and the second housing components can be provided.

With a further example the flank angle of the second tooth flank and/or the flank angle of the second flank is greater than or equal the thread pitch of the helical thread and/or of the helical counter thread of the threaded connection. In this way a comparatively large release force can be implemented.

Here, and for comparing the steepness of the tooth flank or flank with the thread pitch the flank angle can be regarded as the ratio between the circumferential extent of the flank versus its longitudinal extent.

According to a further example the toothed profile comprises a number of locking teeth and locking recesses in an alternating arrangement along a circumference of the first housing component. The toothed profile may comprise an equal number of locking teeth and locking recesses. As seen in circumferential direction a locking tooth follows a locking recess and a locking recess follows a locking tooth, and so on. Hence, the entirety of a circumference of the first housing component can be provided with an alternating sequence of locking teeth and locking recesses. With some examples a locking tooth circumferentially directly adjoins a locking recess and a locking recess circumferentially directly adjoins a locking tooth.

With some other examples there may be provided a circumferential or tangential gap between a locking tooth and a locking recess on one of the first and second housing components. Here, the tangential or circumferential offset of the locking teeth and locking recesses of the toothed profile is complementary shaped to the counter toothed profile in order to enable and to provide a smooth mutual engagement of the respective profiles in the final assembly configuration.

Accordingly, and with a further example, the counter toothed profile comprises a number of locking recesses and locking teeth in an alternating arrangement along a circumference of the second housing component. Typically, the counter toothed profile is complementary shaped to the toothed profile. Insofar, the same features and effects as described above with regards to the numerous locking teeth and locking recesses provided on the toothed profile equally apply to the counter toothed profile.

By increasing the number of locking teeth and complementary shaped locking recesses on the locking structure and the counter locking structure the magnitude of an interlock force and the magnitude of a release force can be modified accordingly. Here, numerous friction forces between individual locking teeth with flanks of complementary-shaped locking recesses contribute to a total force or torque required to establish or to release the threaded connection between the first and the second housing components. By increasing or decreasing the number of locking teeth and locking recesses the magnitude of respective forces or torques can be the varied.

In effect, and with some examples the toothed profile may comprise a saw tooth profile extending along the circumference of the sidewall of the first housing component. Correspondingly, the counter toothed profile may comprise a counter saw tooth profile extending along the circumference of the sidewall of the second housing component. The teeth of the toothed profile typically extent or protrude in longitudinal or axial direction so as to engage with complementary shaped recesses of the respective counter toothed profile.

According to a further example the first housing component comprises a proximally facing stop face provided with a toothed profile. The second housing component comprises a distally facing counter stop face provided with a counter toothed profile. Typically, the first housing component is subject to a proximally directed motion relative to the second housing component when the insert is inserted into the receptacle. Here, and with some examples the proximally facing stop face comprises the toothed profile of the locking structure and the distally facing counter stop face comprises the counter toothed profile of the counter locking structure.

By providing a proximally facing stop face on the first housing component the insertion of the insert into the receptacle can be effectively blocked or stopped when reaching the final assembly configuration.

When reaching a final assembly configuration, the proximally facing stop face axially or longitudinally engages or axially abuts the distally facing counter stop face. Since the assembly process is governed by a helical motion of the first and second housing components relative to each other the axial or longitudinal abutment is reached through a kind of a ratchet or snap engagement of the locking structure with the counter locking structure.

In this way and by providing a threaded connection in combination with a rotation lock the axial or longitudinal abutment of the proximally facing stop face with the distally facing counter stop face provides an immediate stop for limiting or locking a further helical and thus combined rotational and longitudinal or axial movement of first and second housing components relative to each other when a final assembly configuration has been reached. At the same time and by way of the rotation lock, a further rotational movement between the first and second housing components is effectively blocked. In this way, the axial abutment between the proximally facing stop face and the distally facing counter stop face is not over stressed.

The rotation lock provides and impedes a helical motion of the first housing component relative to the second housing component beyond the final assembly configuration. In this way, the rotation lock serves to prevent over stressing the mutual longitudinal abutment between the distally facing counter stop face and the proximally facing stop face of the first and second housing components, respectively. Hence, a damaging of the first or second housing components due to over stressing the threaded connection can be effectively prevented.

According to a further example, one of the proximally facing stop face and the distally facing counter stop face is a longitudinal end face of one of the first housing component and the second housing component. The other one of the proximally facing stop face and the distally facing counter stop face is provided on a stepped portion radially protruding from the respective sidewall. The stepped portion may protrude radially inwardly or outwardly from a sidewall of the other one of the first housing component and the second housing component. The respective stop face faces in distal or proximal direction on the stepped portion on one of an outside surface or inside surface of the sidewall of the other one of the first housing component and the second housing component.

With some examples and when for instance the proximally facing stop face is provided at a longitudinal end, hence at a proximal end of the insert the distally facing counter stop face is located on a stepped portion protruding radially inwardly from an inside surface of the sidewall of the receptacle. The distally facing counter stop face is hence provided on a radially stepped down portion of the inside sidewall of the receptacle.

With other examples and when for instance the distally facing counter stop face is provided as a longitudinal end of the receptacle the proximally facing stop face is provided on a radially outwardly stepped portion of the insert. Here, the radially outwardly stepped portion may be even provided at a longitudinal end of the insert, e.g. at a distal end of the insert, which is separated from an end face of the insert by the axial or longitudinal length of the insert.

In this way, a nested arrangement of first and second housing components or a kind of a telescopic arrangement of the insert inside the receptacle can be provided. In particular and when the at least one of the proximally facing stop face and the distally facing counter stop face is provided on a longitudinal end face or longitudinal end of the insert the rotation lock provided by the locking structure and the complementary-shaped counter locking structure is invisible from outside the housing when the first and second housing components are in a final assembly configuration.

With other examples and when for instance one of the proximally facing stop face and the distally facing counter stop face is provided at a longitudinal end face of the receptacle the rotation lock is visible from outside. This way, a mutual engagement of the locking structure and the counter locking structure and establishing of the rotation lock between first and second housing components can be visually inspected.

According to a further example the proximally facing stop face is located on an annular rim protruding radially outwardly from a longitudinal end of the insert. The longitudinal end of the insert may be the distal end of the insert, while the proximal end of the insert may form a free end of the respective connecting end of the housing component. Hence, the proximally facing stop face and hence the annular rim is longitudinally offset from a free end of the insert. The axial or longitudinal separation coincides with the respective axial or longitudinal extent of the insert.

With some examples the helical thread provided on the insert comprises at least one or more complete convolutions. Correspondingly, also the helical counter thread provided on the inside of the sidewall of the receptacle comprises one or more complete convolutions. By implementing a threaded connection based on one or more convolutions of the respective helical structure, e.g. based on at least two, at least three, at least four or at least five complete convolutions, a rather precise and robust threaded connection between the first and second housing components can be provided. This enables a rather tilt-free and/or slack-free screwed connection of the first and the second housing components. Such a tilt- and/or slack-free mutual engagement is of particular benefit to establish a robust and precise rotation lock.

According to a further example there is provided a first indicator on an outside surface of the first housing component and there is provided a second indicator on an outside surface of the second housing component. Typically, the first or second indicator is located longitudinally offset from the insert of the respective first or second housing component. The first indicator and the second indicator are implemented as visible and/or palpable indicators.

The first indicator and the second indicator may align in longitudinal direction when the first and second housing components have reached the final assembly configuration. In this way the first and second indicators may provide a visual and/or palpable guiding for establishing the screw type connection between the first and the second housing components.

With some examples and when the helical thread comprises an integer number of convolutions and/or when the helical counter thread comprises an integer number of convolutions the first indicator and the second indicator may be longitudinally aligned during or after longitudinally inserting the insert into the receptacle. In this way and when the first indicator is aligned with the second indicator the helical thread may directly engage the helical counter thread and the user is no longer obliged to rotate the first housing component relative to the second housing component in an unscrewing direction until the helical thread engages the helical counter thread.

In this way the first indicator and the second indicator on the first and second housing components, respectively, may provide a twofold or double function. They may indicate the orientation of the first housing component relative to the second housing component in order to engage the helical thread with the helical counter thread and further to indicate that first and second housing components have reached a final assembly configuration at the end of a screwing down assembly of the first and second housing components.

According to a further example at least one of the proximally facing stop face and the distally facing counter stop face is located on an annular rim provided on the respective first or second housing component. The annular rim may protrude radially from a sidewall of the first housing component or second housing component. The annular rim may protrude radially outwardly from an outer surface of the first or second housing component. It may protrude radially inwardly from an inside surface of the first or second housing component. The annular rim protruding radially inwardly and/or radially outwardly from an inside surface or outside surface of the first and/or second housing component may provide a structural strengthening and stiffening of the respective housing component, in particular in a region or section of the housing component that is provided with the rotation lock.

In this way, a particular portion of the first or second housing component provided with the rotation lock and hence provided with a locking structure or counter locking structure can be structurally strengthened. With some examples the annular rim and the sidewall of the first housing component and/or the sidewall of the second housing component are integrally formed. Typically, the first housing component and/or the second housing component may comprise an injection molded plastic component. With other examples the annular rim may be implemented as an insert or as a separate piece to be assembled with the respective housing components. It may be clamped into or clamped onto the respective housing component. With other examples the annular rim may be fastened to at least one of the first and second housing components, e.g. by a friction fit, by an adhesive, by welding or by combinations thereof.

According to a further example the housing comprises a mechanical coding provided on the insert and further comprises a mechanical counter coding provided in the receptacle. The mechanical coding is complementary or correspondingly shaped to the mechanical counter coding and may form a matching pair of a coding and a counter coding of a common type. A mechanical coding and a mechanical counter coding of different types are operable and configured to prevent a mutual assembly of the first housing component and the second housing component in the final assembly configuration. Here, and with a non-matching pair of a coding and a counter coding, the mechanical coding and the mechanical counter coding may be operable to prevent establishing of a threaded connection between the first housing component and the second housing component.

Generally, a mutual assembly of the first housing component and the second housing component and/or a threaded connection between the first housing component and the second housing component requires that a first housing component provided with a mechanical coding is assembled with a second housing component provided with a corresponding or complementary shaped mechanical counter coding.

In this way different housings for different drug delivery devices can be provided having first and second housing components that are of similar or even identical outer shape but distinguish by way of a coding and a counter coding. In this way, unintended cross use of e.g. a first housing component of a housing of a first type with a second housing component of a housing of a second type can be effectively prevented. It is only when the mechanical coding of the insert matches the mechanical counter coding in the receptacle that the threaded connection between first and second housing components can be established to mutually connect and to mutually fix the first and second housing components. With all other pairings or combinations of a mechanical coding, e.g. a mechanical coding of a first type with a non-matching mechanical counter coding, e.g. of a second type, the threaded insert cannot engage with the counter threaded receptacle. Then, the mutual assembly and/or fixing of the first and the second housing component is effectively prevented.

Prevention of the mutual engagement of the first connecting end with the second connecting end of non-matching housing components can be effectively achieved in at least two different ways. According to some examples the mechanical coding and the non-matching mechanical counter coding are configured to prevent at least a partial insertion or a complete insertion of the insert into the receptacle along the longitudinal direction. Here, and with a coding on the insert non-matching with the counter coding of the receptacle the insert may be mechanically blocked from entering the receptacle. Alternatively, the insert may be sized and shaped to enter the receptacle but then the coding non-matching with the counter coding is configured to prevent an engagement of the helical thread with the helical counter thread. In effect, for a coding non-matching with a counter coding the first housing component cannot be connected or fixed to the second housing component.

According to a further example the mechanical coding comprises a coding feature integrated into the helical thread. The mechanical counter coding comprises a counter coding feature integrated into the helical counter thread. The coding feature and the counter coding feature may distinguish by their diameter, by the type and/or or by the profile of the helical thread and the helical counter thread, respectively. Only a helical thread and hence only a coding feature mechanically matching the helical counter thread and hence matching the counter coding feature may provide a mutual connection and fixing of first and second housing components.

According to another example the coding feature is defined by at least one of a thread type, a threaded profile and a thread pitch of the helical thread. Correspondingly, the counter coding feature is defined by at least one of a thread type, a thread profile and a thread pitch of the helical counter thread. The coding feature only matches the counter coding feature if the thread type, the thread profile and the thread pitch of the helical thread matches the respective thread type, the thread profile and the thread pitch of the helical counter thread; and vice versa. All other combinations, wherein at least only one of the thread type, the thread profile and the threaded page of the helical thread does not match with a respective thread type, thread profile or thread pitch of the helical counter thread will prevent a mutual engagement of the helical thread and the helical counter thread. Rather, with such combinations a mutual engagement of the helical thread with the helical counter thread is effectively prevented and impeded.

For instance, at least one of the helical thread and the helical counter thread comprises one of the following thread types, namely, a pipe thread, a trapezoidal thread, a knuckle thread, a Whitworth thread or a buttress thread.

According to another aspect there is provided an injection device for injecting a dose of a medicament. The injection device comprises a housing as described above and a cartridge arranged inside the housing. The cartridge comprises a barrel filled with a medicament and sealed in a proximal longitudinal direction by a movable bung. The injection device further comprises a drive mechanism arranged inside the housing. The drive mechanism comprises a piston rod operable to exert a distally directed dispensing force onto the bung of the cartridge. Typically, the injection device is implemented as a hand held or portable injection device. The injection device may comprise a pen-type injector.

With some examples the receptacle is provided as a housing insert fixedly attachable or fixedly attached to an elongated housing component, e.g. the first or second housing component of the housing of the drug delivery device. The housing insert may be rotationally and/or longitudinally fixed to the elongated housing component. Insofar all features and benefits as described above in connection with the receptacle equally apply to a housing insert fixedly connectable or fixedly connected to a respective housing component.

In another aspect the disclosure relates to a housing of drug delivery device, e.g. of an injection device. Also here, the housing comprises a first housing component configured to accommodate a cartridge filled with a medicament. The first housing component comprises a first connecting end. The housing further comprises a second housing component. The second housing component is configured to accommodate a drive mechanism of the drug delivery device. Typically, the drive mechanism comprises a piston rod extending in longitudinal direction and configured to operably engage with a piston or bung of the cartridge for expelling a dose of the medicament from the cartridge.

The second housing component comprises a second connecting end. Typically, the first connecting end is connectable to the second connecting end to form or to constitute the housing of the drug delivery device. There is further provided an insert on one of the first connecting end and the second connecting end. The insert is typically integrally formed with the respective first or second housing component. There is further provided a receptacle on the other one of the first connecting end and the second connecting end. The insert is insertable into the receptacle along the longitudinal direction for mutually fastening the first housing component and the second housing component and/or for forming or establishing the housing of the drug delivery device. Typically, the receptacle is provided at one of the first and second connecting ends and forms a respective connecting end. The insert is provided on the other one of the first and second connecting ends and forms a respective connecting end.

The receptacle comprises an inner cross-section sized and shaped to receive the insert therein. Typically, an inside diameter or inside cross-section of the receptacle closely matches an outside diameter or outer cross-section of the insert.

The housing may further comprise a fastening element provided on the insert and a counter fastening element complementary shaped to the fastening element and provided in the receptacle. Typically, and when reaching a final assembly configuration, the fastening element engages the counter fastening element thereby fastening and fixing the first housing component to the second housing component; and vice versa.

The housing further comprises a threaded connection to mutually connect and/or to mutually fix the first connecting end and the second connecting end. The threaded connection comprises a helical thread provided on the insert. The threaded connection further comprises a helical counter thread complementary shaped to the helical thread and provided in the receptacle. The helical thread is typically provided on an outside surface of the insert. The helical counter thread is typically provided on an inside surface of a sidewall of the receptacle. Typically, one of the helical thread and the helical counter thread comprises a radially protruding rib and the other one of the helical thread and the helical counter thread comprises a radially recessed groove complementary shaped to the radially protruding rib.

The fastening element of the insert may be provided by the helical thread and the counter fastening element of the receptacle may be provided by the helical counter thread.

The housing further comprises a mechanical coding provided on the insert and further comprises a mechanical counter coding provided in the receptacle. The mechanical coding is complementary or correspondingly shaped to the mechanical counter coding and may form a matching pair of a coding and a counter coding of a common type. A mechanical coding and a mechanical counter coding of different types are operable and configured to prevent a mutual assembly of the first housing component and the second housing component in the final assembly configuration. Here, and with a non-matching pair of a coding and a counter coding, the mechanical coding and the mechanical counter coding may be operable to prevent establishing of a threaded connection between the first housing component and the second housing component. Generally, a mutual assembly of the first housing component and the second housing component and/or a threaded connection between the first housing component and the second housing component requires that a first housing component provided with a mechanical coding is assembled with a second housing component provided with a corresponding or complementary shaped mechanical counter coding.

Generally, the mechanical coding and the mechanical counter coding can be implemented as described above.

The housing according to the present aspect may be void of a rotation lock. Here, the rotation lock and any of the features and effects as described above in connection with the rotation lock may be provided and implemented optionally.

According to another aspect the disclosure relates to a kit of at least a first housing as described above and a second housing as described above. The coding feature of the first housing distinguishes from the coding feature of the second housing thing with regard to at least one of a thread type, a thread profile and a thread pitch of the helical thread.

Correspondingly, the counter coding feature of the first housing distinguishes from the counter coding feature of the second housing with regard to at least one of a thread type, a thread profile and a thread pitch of the helical counter thread.

According to a further example at least one of a thread type, a thread profile and a thread pitch of the helical thread of a first housing component of a first housing of a kit of numerous housings of respective injection devices distinguishes from and does not match at least one of a respective thread type, a thread profile and a thread pitch of the helical counter thread of the second housing component of a second housing. In this way the first housing component of the first housing is unable to pair or to connect with the second housing component of the second housing. Unintended cross-use of first and second housing components provided and equipped with different and hence non-matching codings and counter codings can be effectively prevented and impeded.

The first housing is provided with a pair of a coding and a counter coding of a first type. The second housing is provided with a pair of a coding and a counter coding of a second type. The coding of the first type is unable to pair or to engage the counter coding of the second type. Vice versa, the counter coding of the first type is unable to pair or to engage with the coding of the second type.

Only the coding of the first type is able and configured to pair or to engage with the counter coding of the first type. The coding of the second type is only and exclusively engageable or connectable to the counter coding of the second type; and vice versa.

Generally, and with some examples the first housing components of different housings may distinguish by the size and/or geometry of an accommodating space for receiving a medicament container or cartridge. In particular, a housing with a coding of a first type may be exclusively equipped with a first cartridge or medicament container. A housing with a coding of a second type may be exclusively equipped with a cartridge or a second medicament container. For this, medicament containers, cartridges as well as the interior of the first housing components may comprise further codings or coding features or may distinguish with regard to their size or geometry such that only one dedicated cartridge or medicament container unequivocally fits into only one dedicated first housing component.

With some examples, the first housing component is provided with a mechanical coding to engage with a complementary shaped counter coding of a cartridge. With further examples the first housing component may be provided with at least one of an electronic, a visual or optical coding configured to match with a complementary counter coding of the cartridge, which is also of electronic, visual or optical type.

Moreover, at least one of the cartridge and the first housing component may be provided with a locking or fastening feature by way of which a cartridge can be fixed and/or retained in the first housing component. Here, the first housing component, e.g. implemented as a cartridge holder, and a cartridge assembled therein can be provided as a pre-fabricated housing assembly or as a dedicated cartridge-cartridge holder combination.

In either way, it can be assured or provided that a particular medicament provided in a particular cartridge is unequivocally associated with a particular type of a first housing component, i.e. with a particularly mechanically encoded first housing component. In effect and with some examples, a cartridge provided with a particular medicament can be only accommodated in a correspondingly shaped first housing component equipped with a respective mechanical coding.

With further examples a pre-fabricated housing assembly or a dedicated cartridge-cartridge holder combination is commercially distributed by a pharmaceutical manufacturer. Here, the cartridge may be undetachably or irremovably fixed inside the first housing component and the pharmaceutical manufacturer provides a respective matching between a cartridge filled with a particular medicament and a suitable first housing component, which is mechanically encoded in accordance to the type of medicament located inside the cartridge.

According to a further aspect the present disclosure also relates to a kit of injection devices. The kit of injection devices comprises at least a first injection device comprising a first housing provided with a coding and a counter coding both of a first type and further comprises a second injection device with a second housing provided and equipped with a coding and a counter coding both of a second type non-matching with the respective counter coding or coding of the first type.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of injection devices with dedicated or coded housing components will be described in greater detail by making reference to the drawings, in which:

FIG. 1 schematically illustrates an example of a drug delivery device,

FIG. 2 shows an example of an exploded view of the drug delivery device of FIG. 1,

FIG. 3 is a schematic exploded view of first and second housing components before assembly,

FIG. 4 shows the first and second housing components during assembly,

FIG. 5 shows the first and second housing components in a final assembly configuration,

FIG. 6 is illustrative of a first connecting end of the first housing component,

FIG. 7 is illustrative of a second connecting end of the second housing component,

FIG. 8 schematically illustrates a mutual arrangement of the first and second housing components during a final step of assembly,

FIG. 9 shows the configuration of FIG. 8 when the first housing component has screwed down slightly further towards the final assembly configuration compared to FIG. 8,

FIG. 10 shows the arrangement of FIG. 9 with the first housing component screwed down further,

FIG. 11 shows the first and second housing components in the final assembly configuration of FIG. 5,

FIG. 12 shows numerous examples of different thread types and different coding features provided on the insert and/or inside the receptacle,

FIG. 13 shows an example of a non-matching pairing of a coding feature with a counter coding feature,

FIG. 14 shows a further example of a non-matching pairing of a coding and a counter coding,

FIG. 15 shows another example of a non-matching pairing of a coding and a counter coding,

FIG. 16 shows another example of a non-matching pairing of a coding and a counter coding, and

FIG. 17 shows another example of a non-matching pairing of a coding and a counter coding.

### DETAILED DESCRIPTION

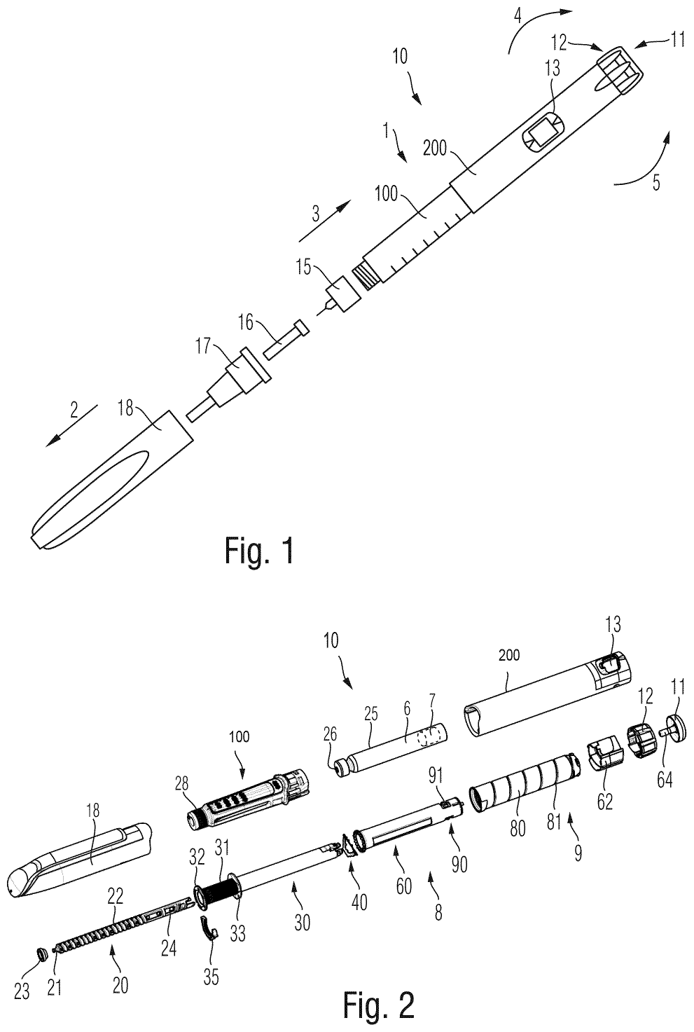

In FIGS. 1 and 2 only one of numerous examples of a handheld injection device is illustrated, that is generally usable in combination with a wearable electronic device. The device as shown in FIGS. 1 and 2 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 comprises a first housing component 100 and a second housing component 200. The second housing component may form a main housing part configured to accommodate a drive mechanism 8 and/or a dose setting mechanism 9 as shown in FIG. 2. The first housing component 100 is configured as a cartridge holder. It may be permanently or releasably connected to the second housing component 200.

The first housing component 100 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder and hence the first housing component 100 comprises a threaded socket 28 at its distal end to threadedly engage with a correspondingly threaded portion of the injection needle 15. By attaching the injection needle 15 to the distal end of the first housing component 100 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 μg of pure crystalline insulin (1/22 mg). The dose dial 12 may comprise or may form a dose dial.

As shown further in FIGS. 1 and 2, the housing 10, e.g. the second housing component 200 comprises a dosage window 13 that may be in the form of an aperture in the housing 10. The dosage window 13 permits a user to view a limited portion of a number sleeve 80 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The click sound is typically generated by a click noise generator 45. Generally, a click noise generator 45 may be implemented in various different ways. The number sleeve 80 mechanically interacts with a piston in the insulin cartridge 6. When the needle 15 is stuck into a skin portion of a patient, and when the trigger 11 or injection button is pushed, the dose displayed in display window 13 will be ejected from injection device 1. When the needle 15 of the injection device 1 remains for a certain time in the skin portion after the trigger 11 is pushed, the dose is actually injected into the patient's body. Ejection of a dose of the liquid medicament may also cause a mechanical click sound, which is however different from the click sound produced when using the dose dial 12. For this, the injection device one may comprise a separate, hence a second click noise generator (not illustrated).

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

An example of the drive mechanism 8 is illustrated in more detail in FIG. 2. It comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10, e.g. to the second housing component 200. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number 80 to prevent relative movement there between. The dose dial 12 is provided with a central opening.

The trigger 11, also denoted as dose button is substantially T-shaped. It is provided at a proximal end of the injection device 1. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40, also acting as a click noise generator 45, and the clutch 60 engaged, the drive sleeve 30, the spring 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 to prevent further movement in a first sense of rotation, e.g. in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose. Here, the clutch may serve as a ratchet mechanism.

As an alternative or in addition the ratchet mechanism 90 may comprise at least one ratchet feature 91, such as a flexible arm on the sidewall of the tubular-shaped clutch 60. The at least one ratchet feature 91 may comprise a radially outwardly extending protrusion e.g. on a free end of the flexible arm. The protrusion is configured to engage with a correspondingly shaped counter ratchet structure on an inside of the number sleeve 80. The inside of the number sleeve 80 may comprise longitudinally shaped grooves or protrusions featuring a saw-tooth profile. During dialing or setting of a dose the ratchet mechanism 90 allows and supports a rotation of the number sleeve 80 relative to the clutch 60 along a second sense of rotation 5, which rotation is accompanied by a regular clicking of the flexible arm of the clutch 60. An angular momentum applied to the number sleeve 80 along the first sense of rotation for is unalterably transferred to the clutch 60. Here, the mutually corresponding ratchet features of the ratchet mechanism 90 provide a torque transmission from the number sleeve 80 to the clutch 60.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position may be determined by the abutment of one of axially extending edges or stops of the number sleeve 80 with at least one or several corresponding stops of the housing 10.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

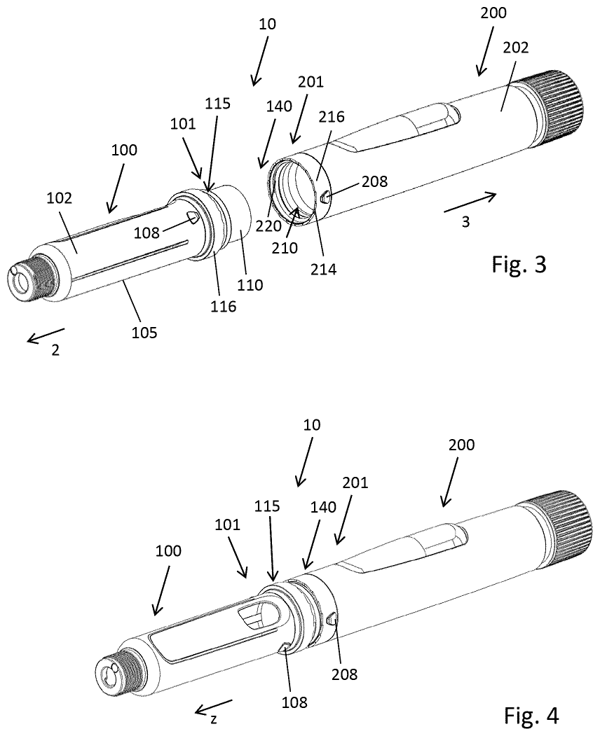

The housing 10 as illustrated in the numerous examples of FIGS. 3-11 comprises a first housing component 100 and a second housing component 200. The first housing component 100 is configured as a cartridge holder. It is sized and shaped to accommodate a cartridge 6 inside its hollow interior. The cartridge holder and hence the first housing component 100 comprises a first connecting end 101. The first connecting end 101 forms a proximal end of the first housing component 100. Correspondingly, the second housing component 200 comprises a second connecting end 201, typically at a distal end of the housing component 200.

The first connecting end 101 is mechanically connectable to the second connecting end 201. As illustrated, the first housing component 100 comprises an insert 110 forming the first connecting end 101. The second housing component 200 comprises a receptacle 210 shaped and sized to receive the insert 110. The insert 110 is at least partially insertable into the receptacle 210 by a longitudinal sliding movement relative to the second housing component 200, in particular along the proximal direction 3.

The insert 110 forms a proximal end of the first housing component 100. The insert 110 comprises a proximal end face 112. Towards the distal direction 2 the insert 110 is confined by a flange section 115 protruding radially outwardly from the tubular shaped sidewall 102 of the first housing component 100 and hence also from a sidewall 102 of insert 110. The flange section 115 is structurally stiffened by a circumferential rim 116 protruding radially outwardly from the outside surface 105 of the sidewall 102 of the first housing component 100.

In other words, the flange section 115 comprises the circumferential rim 116 extending all around the tubular shaped insert 110. Towards the proximal direction 3 the flange section 115 comprises an abutment or stop face 114 facing in proximal direction 3. The abutment or stop face 114 is configured to axially abut a distal end face 214 of a sidewall 202 of the second housing component 200. The sidewall 102 of the first housing component 100 may comprise a window 103, which can be implemented as a through recess intersecting the sidewall 102. The window 103 allows and supports visual inspection of the cartridge 6 and its content arranged inside the first housing component 100.

The receptacle 210 is open towards the distal direction 2. Insofar, the insert 110 of the first housing component 100 can be inserted into the receptacle 210 along the proximal direction 3 until a final assembly configuration has been reached. There is provided a fastening element on the outside surface 105 of the insert 110 complementary shaped to a counter fastening element provided on an inside surface 203 of the sidewall 202 of the receptacle 210.

With the present example the fastening element and the complementary shaped counter fastening element form or constitute a threaded connection 140 for the first connecting end 101 and the second connecting end 201. As illustrated in greater detail in FIGS. 6 and 7 the first connecting end 101 is provided with a helical thread 120 on the outside surface 105 of the insert 110. The receptacle 210 comprises a complementary shaped helical counter thread 220 provided on the inside surface 203 of the sidewall 202 of the receptacle 210. The helical thread 120 comprises at least one, preferably numerous complete, e.g. an integer number of convolutions. The helical counter thread 220 is complementary shaped and comprises also at least one or numerous convolutions.

The helical thread 120 is provided at a proximal end of the insert 110 and longitudinally adjoins the flange section 115. In this way the insert 110 comprises a proximal section 111 longitudinally offset from the flange section 115. In the presently illustrated example, the proximal section 111 is void of radial protrusions or recesses and is complementary shaped to an inside surface 203 of the sidewall 202 of the receptacle 210. This enables and provides a smoothly sliding inserting motion of the insert 110 into the receptacle 210. The rather evenly shaped proximal section 111 of the insert 110 may be radially and longitudinally guided by the complementary shaped inside surface 203 of the sidewall 202. Insofar, the outer diameter of the proximal section 111 may closely match a respective inside diameter of the sidewall 202 of the receptacle 210.

The helical thread 120 protrudes radially outwardly from the proximal section 111. However, it is located radially inwardly from the flange section 115. The helical thread 120 is located longitudinally between the proximal section 111 and the radially outwardly protruding flange section 115. The helical thread 120 may longitudinally adjoin the radially widened or radially outwardly protruding flange section 115. The flange section 115 may be structurally strengthened by the radially outwardly protruding circumferential rim 116. The rim 116 is provided with the proximally facing stop face 114 configured to abut or to engage with the complementary shaped distally facing stop face or end face 214 of the sidewall 202 of the receptacle 210 of the second housing component 200. Also, the stop face 214 may be provided on a structurally stiffened or stiffening rim 216 arranged on or integrally formed with the sidewall 202 of the second housing component 200. As shown in FIG. 7 the rim 216 may be arranged on the outside surface 205 of the sidewall 202 of the second housing component 200. It may protrude radially outwardly from the sidewall 202 and may form or constitute the distal end face 214 of the sidewall 202.

The counter thread 220 is provided near and/or longitudinally adjacent an insert opening 211 of the receptacle 210. In this way it can be provided that in a first step of assembly the proximal section 111 of the insert 110 is inserted into the receptacle 210 at least to a certain extent before the helical thread 120 engages the complementary shaped helical counter thread 220. Starting from such an intermediate assembly configuration the further assembly process of the first housing component 100 and the second housing component 200 requires a twisting motion of the first housing component 100 relative to the second housing component 200 in accordance to the helical shape of the thread 120 and the complementary shaped counter thread 220 until a final assembly configuration has been reached.

The final assembly configuration is characterized by the stop face 114 engaging the complementary shaped counter stop face 214. The proximally facing stop face 114 may then axially and/or longitudinally engage or abut with the complementary shaped counter stop face 214.

As further indicated in the sequence of FIGS. 3-7 there is provided a first indicator 108 on the outside surface 105 of the sidewall 102 of the first housing component 100. There is also provided a complementary or correspondingly shaped second visual indicator 208 on the outside surface 205 of the sidewall 202 of the second housing components 200. The first visual indicator 108 is located adjacent the flange section 115 but longitudinally offset from the insert 110. The second visual indicator 208 is provided near a distal end of the sidewall 202. The visual indicators 108, 208 may protrude radially outwardly on the outside surface 105, 205 of the housing components 100, 200. In this way, they are even palpable and may be useful for persons being visually impaired.

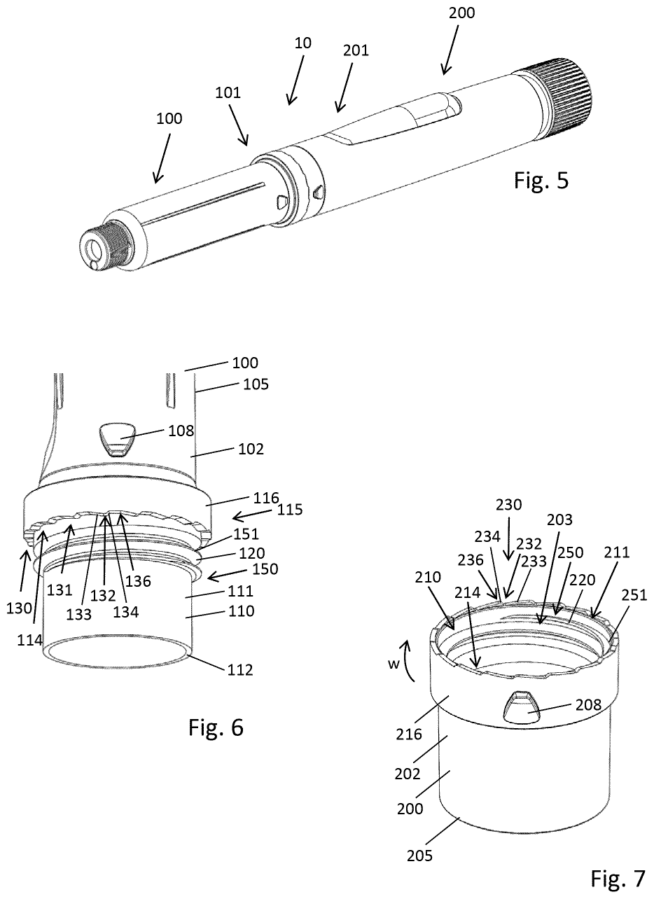

The visual indicators 108 and 208 align along the longitudinal direction (z) as indicated in FIG. 5 when a final assembly configuration has been reached and hence when the first housing component 100 and the second housing component 200 have been screwed down by the threaded connection 140, i.e. by the mutually engaging arrangement of the helical thread 120 with the helical counter thread 220.

When the helical thread 120 and the helical counter thread 220 comprise an integer number of convolutions the first and second indicators 108, 208 may also indicate a mutual orientation of the first and second housing components 100, 200 that is required for the helical thread 120 entering the helical counter thread 220 at the beginning or before the first and second housing components 100, 200 are to be screwed down.

The housing 10 is further provided with a rotation lock 160 that is configured to impede screwing down and/or unscrewing of the first housing component 100 and the second housing component 200 when or after a final assembly configuration of the first and second housing components 100, 200 has been reached. The rotation lock 160 comprises a locking structure 130 on the first housing component 100 and a complementary-shaped counter locking structure 230 on the second housing component 200.

With the presently illustrated example of FIGS. 6 and 7 the locking structure 130 is provided on the proximally facing stop face 114 of the first housing component 100 and the counter locking structure 230 is provided on the complementary shaped distally facing counter stop face 214 provided on or integrated into the second housing component.

The locking structure 130 comprises a toothed profile 131 complementary shaped to a counter toothed profile 231 of the counter locking structure 230. As further illustrated in FIGS. 6-11 the locking structure 130 and the toothed profile 131 comprise at least one locking tooth 132 protruding or pointing in proximal direction 3. The locking tooth 132 is asymmetric at least as seen in circumferential direction w of the tubular shape of the first or second housing components 100, 200. The locking tooth 132 comprises a first tooth flank 133 facing in a first circumferential direction w1. The locking tooth 132 further comprises a second tooth flank 134 facing in the opposite circumferential direction, hence along a second confidential direction w2 opposite to the first circumferential direction w1.

The counter toothed profile 231 comprises at least one complementary shaped locking recesses 232. Also, the recess 232 is of asymmetric profile or asymmetric shape as seen in the circumferential direction w. The locking recess 232 also comprises a first flank 233 extending from a bottom of the recess 232 towards or along the first circumferential direction w1. The locking recess 232 further comprises a second flank 234 extending from a bottom of the locking recess 232 towards or along the second circumferential direction w2. The first tooth flank 133 distinguishes from the second tooth flank 134 by at least one of its circumferential extent and its flank angle, in particular by the magnitude of the flank angle. As illustrated in FIG. 6 the flank angle of the first tooth flank 133 is smaller than a flank angle of the second tooth flank 134. Hence, the second tooth flank 134 is steeper than the first tooth flank 133.

Complementary or accordingly, the circumferential extent of the first tooth flank 133 is larger than the tangential extent of the second tooth flank 134. Likewise, the first flank 233 of the locking recess 232 comprises a circumferential extent that is larger than the circumferential extent of the second flank 234 of the locking recess 232. Accordingly, the second flank 234 is steeper than the first flank 233.

This mutually corresponding but asymmetric shape of the toothed profile 131 and the counter toothed profile 231 provides different torque or forces for establishing and for releasing the rotation lock 160 at the end of an assembly process of first and second housing components.

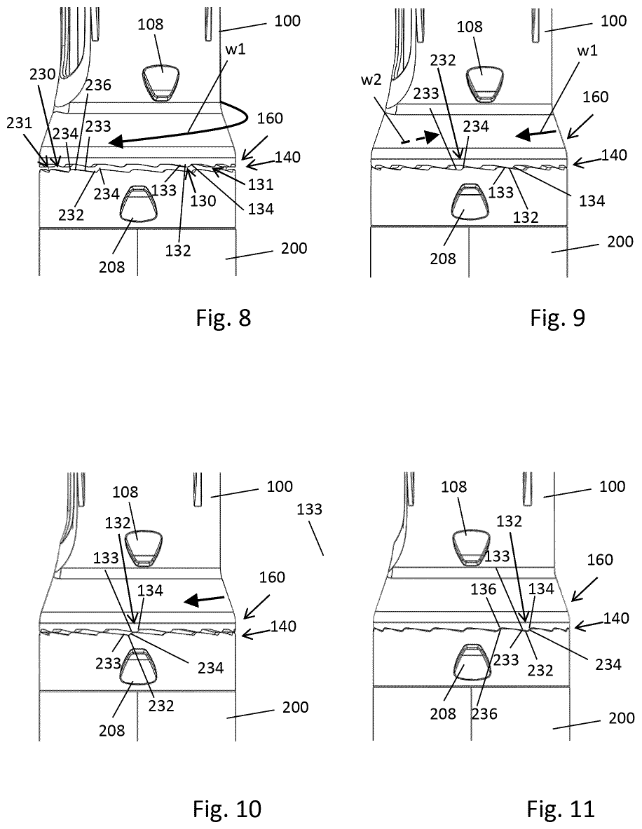

With the presently illustrated example and during the assembly process the first housing component 100 is subject to a helical motion relative to the second housing component 200 along the first circumferential direction w1. As illustrated by the sequence of FIGS. 8-11 only the first tooth flank 133 engages and slides along the first flank 233 as the locking structure 130 and counter locking structure 230 mutually approach. Due to a comparatively low or small flank angle of the first tooth flank 133 and the first flank 233 a further screwing down motion of first and second housing component is still possible even when the tip or crest of the at least one locking tooth 132 gets in mechanical sliding contact with the first flank 233 of the counter toothed profile 231.

Even when the crest of the locking tooth 132 is in sliding contact with the first flank 233 a further helical movement of the first housing component 100 relative to the second housing component 200 along the first circumferential direction w1 is still possible while the toothed profile 131 and the counter toothed profile 231 become subject to an elastic deformation. The elastic deformation which is due to the mutual longitudinal engagement of the toothed profile 131 and the counter toothed profile 231 before reaching the final assembly configuration starts to impede the helical screwing down motion of the first and second housing components 100, 200. Correspondingly, the force or torque for establishing the threaded connection gradually increases.

In the configuration as illustrated in FIG. 10, the force or torque necessary to move the first housing component 100 any further relative to the second housing component 200 is almost at a maximum value. Here, a longitudinal or axial load due to the threaded engagement of the first and the second housing component resulting in a respective compressive force between the toothed profile 131 and the counter toothed profile 231 is comparatively high.

When the first housing component 100 is rotated further along the first circumferential direction w1 relative to the second housing component 200 the locking tooth 132 snaps into and thus engages with a subsequent locking recess 232 as illustrated in FIG. 11. The locking tooth 132 may audibly or haptically engage with the respective locking recess 232. The elastically deformed profiles 131, 231 may relax into an initial state, in which the at least one locking tooth 132 with its first and second tooth flanks 133, 134 is in abutment or tight engagement with the complementary shaped locking recess 232 and its respective flanks 233, 234. At the same time and when reaching a final assembly configuration as illustrated in FIG. 11 the first and second indicators 108, 208 mutually align.

As becomes further apparent from FIGS. 6-11 the locking structure 130 and hence its toothed profile 131 comprises an alternating sequence or alternating arrangement of locking teeth 132 and locking recesses 136. Also, the counter toothed profile 231 comprises a correspondingly or complementary shaped alternating sequence or alternating arrangement of locking recesses 232 and locking teeth 236. As illustrated in greater detail by the sequence of FIGS. 8-11 numerous locking teeth 132 of the toothed profile 131 simultaneously engage with complementary shaped locking recesses 232 of the counter toothed profile 231 upon reaching the final assembly configuration as illustrated in FIG. 11. At the same time or concurrently, numerous locking recesses 136 of the toothed profile 131 mechanically engage with complementary shaped locking teeth 236 of the counter toothed profiles 231.

As further indicated in FIGS. 6-11 the circumferential extent or circumferential length of the first tooth flank 133 substantially equals the circumferential or tangential extent or length of the complementary shaped first flank 233. The same applies to the circumferential or tangential extent of the second tooth flank 134 and the second flank 234. Likewise, also the flank angle of the first tooth flank 133 is substantially equal to the flank angle of the first flank 233. Additionally, the flank angle of the second tooth flank 134 is substantially equal to the flank angle of the second flank 234.

When in the final assembly configuration as illustrated in FIG. 11 the toothed profile 131 is tightly and mechanically engaged with the counter toothed profile 231. Due to the asymmetric shape of the locking teeth 132, 236 and locking recesses 232, 136 an interlock force or torque required to bring the toothed profile 131 and the counter toothed profile in mechanical engagement, e.g. to establish a snap fit engagement of the locking structure 130 with the counter locking structure 230, is less than a release force or torque necessary to disconnect the first and the second housing component, hence to unscrew the first and second housing components 100, 200. During a screwing down of the threaded connection 140 there will be exclusively only the first tooth flank 133 of the toothed profile 131 that slides along the complementary shaped first flank 233 of the counter toothed profile 231.

When in the final assembly configuration and for unscrewing the first and the second housing components the comparatively steep second tooth flank 134 has to slide along the second flank 234. For such a sliding or twisting motion a comparatively high release force or torque has to be applied onto the first housing component 100 along the second circumferential direction w2 relative to the second housing component 200.

The asymmetric profile of the locking teeth 132, 236 provides a twofold rotation lock 160 for the threaded connection 140 of the first and the second housing components 100, 200. It serves to impede over tightening of the threaded connection 140 during assembly of the first and second housing components 100, 200. The rotation lock further provides a well-defined release force for unscrewing of the first and the second housing components from the final assembly configuration as illustrated in FIG. 11.

The housing 10 is further provided with a mechanical coding 150 provided on the insert 110 and further with a mechanical counter coding 250 provided in the receptacle 210. The mechanical coding 150 and the mechanical counter coding 250 are operable to prevent a mutual assembly of the first housing component 100 and the second housing component 200 when the first mechanical coding 150 does not match the counter coding 250. The mechanical coding 150 comprises a coding feature 151 which is integrated into the helical thread 120. The mechanical counter coding 250 comprises a counter coding feature 251 which is integrated into the helical counter thread 220.

In this way there can be provided a kit of numerous housings 10, each of which comprising a first housing component 100 and a second housing components 200. The kit of numerous housings 10 comprises a first housing 10 with a mechanical coding 150 and a mechanical counter coding 250 of a first type and further comprises at least a second housing 10 with a mechanical coding 150′ and a mechanical counter coding 250′ of a second type. The housings are configured such, that a mechanical coding of a particular type exclusively matches a mechanical counter coding of the same type.

Any other combination of a mechanical coding 150 and a mechanical counter coding 250, e.g. a combination of a mechanical coding of a first type with a mechanical counter coding of another type is an incompatible combination. With an incompatible combination or pairing of first and second housing components, a mutual assembly of first and second housing components is prevented. For example, a first housing component of a first housing provided with a mechanical coding 150 of a first type is unable to engage or to connect with a second housing component of a second housing provided with a mechanical counter coding 250′ of a second type; and vice versa.

The coding feature 151 is defined by at least one of a thread type, a thread profile and a thread pitch of the helical thread 120 or by combinations thereof. Correspondingly, the counter coding feature 251 is defined by at least one of a thread type, a thread profile and a thread pitch of the helical counter thread 220 or by combinations thereof.

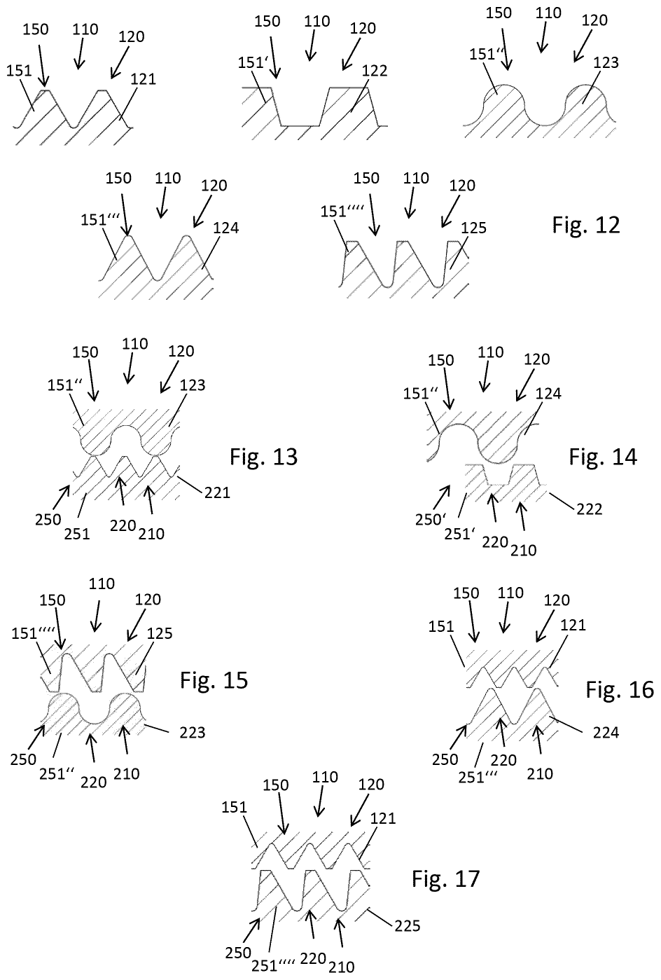

In FIG. 12, numerous examples of different thread types are illustrated. Here, a first thread type 121 representing a first coding feature 151 comprises a pipe thread. A further thread type 122 representing a coding feature 151′ of a second type is implemented as a trapezoidal thread. A third thread profile 123 representing a coding feature 151″ of a third type comprises a knuckled thread. A fourth thread profile 124 representing a coding feature 151′″ of a fourth type comprises a Whitworth thread. A fifth thread profile 125 representing a coding feature 151″″ of a fifth type comprises a buttress thread.

It should be noted that the present illustration of numerous thread types 121, 122, 123, 124, 125 representing different types of coding features 151, 151′, 151″, 151′″, 151″″ is only exemplary. For each one of the numerous thread types 121, 122, 123, 124, 125 there is typically provided also a complementary shaped counter thread type, 221, 222, 223, 224, 225 each of which representing or comprising a respective counter coding feature 251, namely a counter coding features 251 of a first type, a counter coding feature 251′ of a second type, a counter coding features 251″ of a third type, a counter coding features 251′″ of a fourth type or a counter coding features 251″″ of a fifth type, and so on.

Correspondingly, and as illustrated in the sequence of FIGS. 13-17 there may be provided numerous types of codings and counter codings, implemented with and integrated into the helical thread 120 and the helical counter thread 220, respectively. As illustrated in FIG. 13 a helical thread 123 of a third type is unable to pair or to engage with a helical counter thread 221 of the first type.

As illustrated in FIG. 14 the helical thread 123 of the third type is unable to engage with the helical counter thread 222 of the second type.

Likewise, and as illustrated in FIG. 15 the helical thread 125 of the fifth type cannot engage the helical counter thread 223 of the third type.

As illustrated in FIG. 16 the helical thread 121 of the first type cannot engage with the helical counter thread 224 of the fourth type and as shown in FIG. 17, the helical thread 121 of the first type cannot engage the helical counter thread 225 of the fifth type.

Here, each thread type 121, 122, 123, 124, 125 represents and defines a mechanical coding of a respective first, second, third, fourth or fifth type. The same is valid for the counter threads 221-225 that distinguish by their thread type.

Alternative to the illustrated variation of the thread type the mechanical codings 150 and mechanical counter codings 250 may also distinguish and vary by at least one of the thread profile and the thread pitch of the respective helical thread or helical counter thread.

According to further examples it is even conceivable that a thread and hence a mechanical coding distinguishes from another thread by the number of threads on the insert 110 or in the receptacle 210. Here, a multi-start or convoluted thread could be implemented for the helical thread and the complementary shaped helical counter thread. Further variations could be also achieved varying the geometry characteristics, such as thread type, thread pitch or flank angle.

With the presently illustrated examples the insert 110 is provided on the first housing component 100 and the receptacle 210 is provided in the second housing component 200. There are numerous further examples conceivable and within the disclosure of the present application, wherein the insert is provided on the second housing component and wherein the correspondingly-shaped receptacle is provided on the first housing component. Likewise, the specific implementation of radially protruding and radially recessed features, as described in connection with the projection and the groove or in connection with the fastening element and counter fastening element may be interchanged and may be thus provided and implemented in an inverted way compared to the presently shown examples.

### REFERENCE NUMBERS

1 injection device
2 distal direction
3 proximal direction
4 dose incrementing direction
5 dose decrementing direction
6 cartridge
7 bung
8 drive mechanism
9 dose setting mechanism
10 housing
11 trigger
12 dose dial
13 dosage window
14 cartridge holder
15 injection needle
16 inner needle cap
17 outer needle cap
18 protective cap
20 piston rod
21 bearing
22 first thread
23 pressure foot
24 second thread
25 barrel
26 seal
28 threaded socket
30 drive sleeve
31 threaded section
32 flange
33 flange
35 last dose limiter
40 spring
60 clutch
62 insert piece
64 stem
80 number sleeve
81 groove
90 ratchet mechanism
91 ratchet feature
100 housing component
101 connecting end
102 sidewall
103 window
105 outside surface
108 indicator
110 insert
111 proximal section
112 end face
114 stop face
115 flange section
116 rim
120 helical thread
121 thread type
122 thread type
123 thread type
124 thread type
125 thread type
130 locking structure
131 toothed profile
132 locking tooth
133 tooth flank
134 tooth flank
136 locking recess
140 threaded connection
150 mechanical coding
151 coding feature
160 rotation lock
200 housing component
201 connecting end
202 sidewall
203 inside surface
205 outside surface
208 indicator
210 receptacle
211 insert opening
214 stop face
216 rim
220 helical counter thread
221 thread type
222 thread type
223 thread type
224 thread type
225 thread type
230 counter locking structure
231 counter toothed profile
232 locking recess
233 flank
234 flank
236 locking tooth
250 mechanical counter coding
251 counter coding feature

1-15. (canceled)

16. A housing of a drug delivery device, the housing comprising:
a first housing component configured to accommodate a cartridge filled with a medicament, the first housing component comprising a first connecting end;
a second housing component configured to accommodate a drive mechanism of the drug delivery device, the second housing component comprising a second connecting end;
an insert provided on one of the first connecting end and the second connecting end;
a receptacle provided on the other one of the first connecting end and the second connecting end, wherein the insert is insertable into the receptacle along a longitudinal direction for mutually fastening the first housing component and the second housing component;
a threaded connection to mutually connect the first connecting end and the second connecting end, the threaded connection comprising a helical thread provided on the insert and a helical counter thread complementary shaped to the helical thread and provided in the receptacle;
a rotation lock configured to impede screwing and/or unscrewing of the first housing component and the second housing component when the housing is in a final assembly configuration, the rotation lock comprising a locking structure on the first housing component and a counter locking structure complementary shaped to the locking structure and provided on the second housing component.

17. The housing according to claim 16, wherein the locking structure and the counter locking structure are mutually engageable through a twisting helical motion of the first housing component relative to the second housing component upon or during reaching the final assembly configuration.

18. The housing according to claim 16, wherein the locking structure comprises a toothed profile complementary shaped to a counter toothed profile of the counter locking structure, and wherein one of the toothed profile and the counter toothed profile comprises at least one locking tooth extending in the longitudinal direction, and wherein the other one of the toothed profile and the counter toothed profile comprises at least one locking recess complementary shaped to the locking tooth.

19. The housing according to claim 18, wherein the at least one locking tooth comprises an asymmetric profile with regard to a circumferential direction of the first housing component or second housing component.

20. The housing according to claim 18, wherein the at least one locking tooth comprises a first tooth flank facing in a first circumferential direction and a second tooth flank facing in a second circumferential direction opposite the first circumferential direction, and wherein the first tooth flank distinguishes from the second tooth flank by at least one of a circumferential length and a flank angle.

21. The housing according to claim 20, wherein a circumferential length of the first tooth flank is larger than a circumferential length of the second tooth flank, and wherein a flank angle of the first tooth flank is smaller than a flank angle of the second tooth flank.

22. The housing according to claim 18, wherein the toothed profile comprises a number of locking teeth and locking recesses in an alternating arrangement along a circumference of the first housing component.

23. The housing according to claim 16, wherein the first housing component comprises a proximally facing stop face provided with a toothed profile, and wherein the second housing component comprises a distally facing counter stop face provided with a counter toothed profile.

24. The housing according to claim 23, wherein one of the proximally facing stop face and the distally facing counter stop face is a longitudinal end face of one of the first housing component and the second housing component, and wherein the other one of the proximally facing stop face and the distally facing counter stop face is provided on a stepped portion protruding radially from a sidewall of the other one of the first housing component and the second housing component.

25. The housing according to claim 23, wherein at least one of the proximally facing stop face and the distally facing counter stop face is located on an annular rim protruding radially from a sidewall of the insert or receptacle.

26. The housing according to claim 16, comprising a mechanical coding provided on the insert and comprising a mechanical counter coding provided in the receptacle, wherein when the mechanical coding does not match the mechanical counter coding the mechanical coding and the mechanical counter coding are operable to prevent a mutual assembly of the first housing component and the second housing component in the final assembly configuration.

27. The housing according to claim 26, wherein the mechanical coding comprises a coding feature integrated into the helical thread, and wherein the mechanical counter coding comprises a counter coding feature integrated into the helical counter thread.

28. The housing according to claim 27, wherein the coding feature is defined by at least one of a thread type, a thread profile and a thread pitch of the helical thread and wherein the counter coding feature is defined by at least one of a thread type, a thread profile and a thread pitch of the helical counter thread.

29. An injection device for injecting a dose of a medicament, the injection device comprising:
a housing comprising:
a first connecting end;
a second connecting end;
an insert provided on one of the first connecting end and the second connecting end;
a receptacle provided on the other one of the first connecting end and the second connecting end, wherein the insert is insertable into the receptacle along a longitudinal direction;
a threaded connection to mutually connect the first connecting end and the second connecting end, the threaded connection comprising a helical thread provided on the insert and a helical counter thread complementary shaped to the helical thread and provided in the receptacle;
a rotation lock configured to impede screwing and/or unscrewing of the first connecting end and the second connecting end when the housing is in a final assembly configuration, the rotation lock comprising a locking structure and a counter locking structure complementary shaped to the locking structure;
a cartridge arranged inside the housing, the cartridge comprising a barrel filled with a medicament and sealed in a proximal longitudinal direction by a movable bung; and
a drive mechanism arranged inside the housing, the drive mechanism comprising a piston rod operable to exert a distally directed dispensing force onto the bung of the cartridge.

30. The injection device of claim 29, comprising a first housing component configured to accommodate the cartridge filled with a medicament, the first housing component comprising the first connecting end and the locking structure.

31. The injection device of claim 30, comprising a second housing component configured to accommodate a drive mechanism of the drug delivery device, the second housing component comprising the second connecting end and the counter locking structure.

32. The injection device of claim 31, comprising a mechanical coding provided on the insert and comprising a mechanical counter coding provided in the receptacle, wherein when the mechanical coding does not match the mechanical counter coding the mechanical coding and the mechanical counter coding being operable to prevent a mutual assembly of the first housing component and the second housing component in the final assembly configuration.

33. An injection device kit comprising:
a first housing and a second housing, wherein each of the first and second housing comprises:
a first connecting end;
a second connecting end;
an insert provided on one of the first connecting end and the second connecting end;
a receptacle provided on the other one of the first connecting end and the second connecting end, wherein the insert is insertable into the receptacle along a longitudinal direction;
a threaded connection to mutually connect the first connecting end and the second connecting end, the threaded connection comprising a helical thread provided on the insert and a helical counter thread complementary shaped to the helical thread and provided in the receptacle;
a rotation lock configured to impede screwing and/or unscrewing of the first connecting end and the second connecting end when the housing is in a final assembly configuration, the rotation lock comprising a locking structure and a counter locking structure complementary shaped to the locking structure;
a mechanical coding provided on the insert,
a mechanical counter coding provided in the receptacle,
wherein when the mechanical coding does not match the mechanical counter coding the mechanical coding and the mechanical counter coding being operable to prevent a mutual assembly of the first connecting end and the second connecting end in the final assembly configuration; and
wherein a coding feature of the mechanical coding of the first housing distinguishes from a coding feature of the mechanical coding of the second housing with regard to at least one of a thread type, a thread profile and a thread pitch of the helical thread.

34. The kit of claim 33, wherein each of the first and second housing comprises a first housing component configured to accommodate a cartridge filled with a medicament, the first housing component comprising the first connecting end and the locking structure.

35. The kit of claim 34, wherein each of the first and second housing comprises a second housing component configured to accommodate a drive mechanism of the drug delivery device, the second housing component comprising the second connecting end and the counter locking structure.
